(19) United States
(12) Patent Application Publication
(10) Pub. No.: US 2006/0171906 A1
(43) Pub. Date: Aug. 3, 2006

(54) Sustained release tooth whitening formulations and systems

(75) Inventors: Parminder Singh, Union City, CA (US); Eun Soo Lee, Redwood City, CA (US); Appala Sagi, Redwood City, CA (US); Mikhail M. Feldstein, Moscow (RU); Danir F. Bairamov, Sunnyvale, CA (US)

Correspondence Address: MINTZ, LEVIN, COHN, FERRIS, GLOVSKY AND POPEO, P.C, 1400 PAGE MILL ROAD, PALO ALTO, CA, 94304-1124, US

(73) Assignees: Corium International, Inc., Redwood City, CA (US); A.V. Topchiev Institute of Petrochemical Synthesis Russian Academy of Sciences, Moscow (RU)

(21) Appl. No.: 11/315,423

(22) Filed: Dec. 21, 2005

Related U.S. Application Data
(63) Continuation of application No. 60/638,835, filed on Dec. 21, 2004.

(51) Int. Cl. A61K 8/22

(52) U.S. Cl. 424/53

(57) ABSTRACT

A new tooth whitening composition provides sustained release of high levels of whitening agent and is moisture-activated without significant swelling. A preferred system for applying the composition to the teeth is flexible, self-adhesive, and well-tolerated by users. In certain embodiments the tooth whitening composition comprises a mixture of tooth whitening agents, with a first whitening agent selected so as to release peroxide gradually upon contact with moisture and produce an alkaline pH, and a second whitening agent selected so as to release peroxide rapidly upon contact with moisture.

### TECHNICAL FIELD

This invention relates generally to tooth whitening, and more particularly relates to sustained release tooth whitening formulations and systems.

### BACKGROUND

Discoloration of the teeth is a common problem, occurring in two out of three adults. Dental discoloration is considered an aesthetic flaw, and can be particularly distressing or troublesome in situations and professions where showing clean and white teeth is essential. A tooth is composed of an inner dentin layer and an outer, protective layer that is composed of hard enamel but slightly porous. The natural color of the tooth is opaque to translucent white or slightly off-white. Staining of teeth arises as a result of exposure to compounds such as tannins and other polyphenols. These compounds become trapped in or bound to the proteinaceous layer on the surface of teeth, and can penetrate the enamel and even the dentin. On occasion, staining can arise from sources within the tooth, such as tetracycline, which may become deposited in the teeth if administered to an individual when young.

Surface staining can usually be removed by mechanical tooth cleaning. However, discolored enamel or dentin is not amenable to mechanical methods of tooth cleaning, and chemical methods, which can penetrate into the tooth structure, are required to remove the stains. The most effective treatments for dental discoloration are compositions containing an oxidizing agent, such as hydrogen peroxide, that is capable of reacting with the chromogen molecules responsible for the discoloration, and rendering them either colorless or water-soluble, or both.

Consequently, tooth whitening compositions generally fall into two categories: (1) gels, pastes, and liquids, including toothpastes that are mechanically agitated at the stained tooth surface in order to affect tooth stain removal through abrasive erosion of surface stains; and (2) gels, pastes, or liquids that accomplish a tooth-bleaching effect by a chemical process while in contact with the stained tooth surface for a specified period, after which the formulation is removed. In some cases, an auxiliary chemical process, which may be oxidative or enzymatic, supplements the mechanical process.

Some dental compositions such as dentrifices, toothpastes, gels, and powders contain active oxygen or hydrogen peroxide liberating bleaching agents. Such bleaching agents include peroxides, percarbonates, and perborates of the alkali and alkaline earth metals or complex compounds containing hydrogen peroxide. Also, peroxide salts of the alkali or alkaline earth metals are known to be useful in whitening teeth.

Of the many peroxides available to the formulator of tooth whitening compositions, hydrogen peroxide (and its adducts or association complexes, such as carbamide peroxide and sodium percarbonate) has been used almost exclusively. The chemistry of hydrogen peroxide is well known, although the specific nature of its interactions with tooth chromogens is poorly understood. It is believed that hydrogen peroxide destroys tooth chromogens by oxidizing unsaturated carbon-carbon, carbon-oxygen, and carbon-nitrogen bonds found in the stain molecules, thus rendering them colorless or soluble.

A related class of compound, the peroxyacids, has been used in laundry detergents to effectively whiten clothes, due primarily to their stability in solution and their specific binding abilities to certain types of stain molecules. A number of stable, solid peroxyacids have been used, including diperoxydodecanoic acid and the magnesium salt of monoperoxyphthalic acid. Other peroxyacids, such as peroxyacetic acid, are available as solutions containing an equilibrium distribution of acetic acid, hydrogen peroxide, peroxyacetic acid, and water. Alternatively, a peroxide donor such as sodium perborate or sodium percarbonate is formulated together with a peroxyacid precursor. Upon contact with water, the peroxide donor releases hydrogen peroxide which then reacts with the peroxyacid precursor to form the actual peroxyacid. Examples of peroxyacids created in situ include peroxyacetic acid (from hydrogen peroxide and tetraacetylethylenediamine) and peroxynonanoic acid (from hydrogen peroxide and nonanoyloxybenzene sulfonate).

Peroxyacids have also been used in oral care compositions to whiten stained teeth. U.S. Pat. No. 5,279,816 describes a method of whitening teeth comprising the application of a peroxyacetic acid-containing composition having an acid pH. EP 545,594 A1 describes the use of peroxyacetic acid in preparing a composition for whitening teeth. The peroxyacetic acid may be present in the composition, or alternatively, may be generated in situ by combining a peroxide source with a peroxyacetic acid precursor during use. For example, U.S. Pat. No. 5,302,375 describes a composition that generates peroxyacetic acid within a vehicle in situ by combining water, acetylsalicylic acid and a water-soluble alkali metal percarbonate.

The most commonly used dental whitening agent is carbamide peroxide. Carbamide peroxide had been used by dental clinicians for several decades as an oral antiseptic, and tooth bleaching was an observed side effect of extended contact time. Over-the-counter compositions of 10% carbamide peroxide are available as GLY-OXIDE® by Marion Laboratories and PROXIGEL® by Reed and Carnrick, which are low-viscosity compositions that must be held in a tray or similar container in order to provide contact with the teeth. A bleaching gel which is able to hold a comfortable-fitting dental tray in position for an extended time period is available under the trademark OPALESCENCE® from Ultradent Products, Inc. in South Jordan, Utah.

In order for such compositions to stay in place, the compositions must be a viscous liquid or a gel. The use of dental trays also requires that the tray be adapted for comfort and fit so that the tray will not exert pressure or cause irritation to the person's teeth or gums. Such whitening compositions necessarily should be formulated so as to be sufficiently sticky and viscous to resist dilution by saliva.

In one method of whitening an individual's teeth, a dental professional will construct a custom made dental bleaching tray for the patient from an impression made of the patient's dentition and prescribe the use of an oxidizing gel to be dispensed into the bleaching tray and worn intermittently for a period of from about 2 weeks to about 6 months, depending upon the severity of tooth staining. These oxidizing compositions, usually packaged in small plastic syringes or tubes, are dispensed directly by the patient into the custom-made tooth-bleaching tray, held in place in the mouth for contact times of greater than about 60 minutes, and sometimes as long as 8 to 12 hours. The slow rate of bleaching is in large part the consequence of the very nature of formulations that are developed to maintain stability of the oxidizing composition.

For example, U.S. Pat. No. 6,368,576 to Jensen describes tooth whitening compositions that are preferably used with a tray so that the composition is held in position adjacent to the person's tooth surfaces to be treated. These compositions are described as a sticky matrix material formed by combining a sufficient quantity of a tackifying agent, such as carboxypolymethylene, with a solvent, such as glycerin, polyethylene glycol, or water.

In another example, U.S. Pat. No. 5,718,886 to Pellico describes a tooth whitening composition in the form of a gel composition containing carbamide peroxide dispersed in an anhydrous gelatinous carrier, which includes a polyol, a thickener, and xanthan gum.

Yet another example is described in U.S. Pat. No. 6,419,905 to Hernandez, which describes the use of compositions containing carbamide peroxide (0.3-60%), xylitol (0.5-50%), a potassium salt (0.001-10%) and a fluorine salt (0.15-3%), formulated into a gel that contains between 0.5 and 6% by weight of an appropriate gelling agent.

A tooth whitening composition that adheres to the teeth is described in U.S. Pat. Nos. 5,989,569 and 6,045,811 to Dirksing. According to these patents, the gel contains 30-85% glycerin or polyethylene glycol, 10-22% urea/hydrogen peroxide complex, 0-12% carboxypolymethylene, 0-1% sodium hydroxide, 0-100% triethanolamine (TEA), 0-40% water, 0-1% flavor, 0-15% sodium citrate, and 0-5% ethylenediaminetetraacetic acid. The preferred gel according to Dirksing has a viscosity between 200 and 1,000,000 cps at low shear rates (less than one 1/seconds), and is sufficiently adhesive so as to obviate the need for a tray.

Currently available tooth-bleaching compositions have a significant disadvantage in that they cause tooth sensitization in over 50% of patients. Tooth sensitivity may result from the movement of fluid through the dentinal tubules, which is sensed by nerve endings in the tooth, due to the presence of glycerin, propylene glycol, and polyethylene glycol in these compositions. This can result in varying amounts of tooth sensitivity following exposure of the teeth to heat, cold, overly sweet substances, and other causative agents.

Prolonged exposure of teeth to bleaching compositions, as practiced at present, has a number of adverse effects in addition to that of tooth sensitivity. These adverse effects include leaching of calcium from the enamel layer at a pH less than 5.5; penetration of the intact enamel and dentin by the bleaching agents and risking damage to pulpal tissue; and dilution of the bleaching compositions with saliva resulting in leaching from the dental tray and subsequent ingestion by the user.

Some oxidizing compositions (generally having relatively high concentrations of oxidizers) are applied directly to the tooth surface of a patient in a dental office setting under the supervision of a dentist or dental hygienist. Theoretically, such tooth whitening strategies yield faster results and better overall patient satisfaction. However, due to the high concentration of oxidizing agents contained in these so called “in-office” compositions, they can be hazardous to the patient and practitioner alike if not handled with care. The patient's soft tissues (the gingiva, lips, and other mucosal surfaces) must first be isolated from potential exposure to the active oxidizing agent by the use of a perforated rubber sheet (known as a rubber dam), so that only the teeth protrude. Alternatively, the soft tissue may be isolated from the oxidizers to be used in the whitening process by covering the soft tissue with a polymerizable composition that is shaped to conform to the gingival contours and subsequently cured by exposure to a high intensity light source. Once the soft tissue has been isolated and protected, the practitioner may apply the oxidizing agent directly onto the stained tooth surfaces for a specified period of time or until a sufficient change in tooth color has occurred. Typical results obtained through the use of an in-office tooth whitener, range from about 2 to 3 shades (as measured with the VITA Shade Guide, VITA Zahnfarbik).

The range of tooth shades in the VITA Shade Guide varies from very light (B1) to very dark (C4). A total of 16 tooth shades constitute the entire range of colors between these two endpoints on a scale of brightness. Patient satisfaction with a tooth whitening procedure increases with the number of tooth shade changes achieved, with a generally accepted minimum change desirable of about 4 to 5 VITA shades.

It is desirable, with respect to dental care products for tooth whitening, to provide dental care products utilizing an adhesive hydrogel that includes a whitening agent for removing stains from an individual's teeth. Compositions that do not require the use of dental trays to provide contact between the bleaching agent and the teeth are particularly desirable. Such products ideally would cause minimal or no tooth sensitivity, would minimize or eliminate leakage of the whitening agent resulting in ingestion by the user or resulting in damage or irritation to the gums or mucous membranes of the mouth, would provide for longer wear duration, sustained dissolution of the tooth whitening agent, improved efficacy, and be well tolerated by users. It would also be desirable to provide a tooth whitening dental care product that is a solid composition and self-adhesive but that does not stick to the fingers of the user, or that is a non-solid (e.g., liquid or gel) and forms a film when dry.

It would also be advantageous to provide a sustained release tooth whitening composition that provides an initial “burst” of whitening agent, e.g., hydrogen peroxide, followed by sustained release of hydrogen peroxides at elevated levels, such that the whitening effect is maximized as well as prolonged. It would additionally be advantageous to provide a tooth whitening composition that is activated only upon contact with moisture, but which does not swell to any appreciable extent during use.

### SUMMARY OF THE INVENTION

It is a primary object of the invention to provide a tooth whitening composition and system that address the above-mentioned needs in the art. The new tooth whitening composition provides sustained release of high levels of whitening agent and is moisture-activated without significant swelling. The preferred system for applying the composition to the teeth, as will be discussed in detail infra, is flexible, self-adhesive, and generally well-tolerated by users.

In a first embodiment, an improved tooth whitening composition is provided that comprises at least one tooth whitening agent, wherein the improvement comprises incorporating a mixture of tooth whitening agents, with a first whitening agent selected so as to release peroxide gradually upon contact with moisture and produce an alkaline pH, and a second whitening agent selected so as to release peroxide rapidly upon contact with moisture.

In another embodiment, a tooth whitening composition is provided that comprises an admixture of:

a first whitening agent that is inert in a dry environment but activated upon contact with moisture to release hydrogen peroxide and produce an alkaline pH;

a second whitening agent that is inert in a dry environment but activated upon contact with aqueous base; and

a water-swellable, water-insoluble polymer.

In this embodiment, a high pH results due to moisture activation, i.e., hydrolysis, of the first whitening agent. The high pH, in turn, can accelerate degradation of the second whitening agent to yield free radicals at a much faster rate. Free radicals react with stained teeth and render stains colorless. The overall result of increased pH is faster whitening. The water-swellable, water-insoluble polymer may be, by way of example, a cellulosic polymer such as a cellulose ester, an acrylic acid and/or acrylate copolymer, or a mixture of such polymers. For instance, a mixture of acrylic acid and/or acrylate copolymers can be advantageously provided by combining an anionic copolymer with a cationic copolymer such that the copolymers ionically associate with each other, yielding a polymer matrix. An ionically bound polymer matrix reduces swelling of the composition in an aqueous environment, and also allows the tooth whitening agents to remain in the composition longer than would otherwise be possible. These compositions generally, although not necessarily, also contain a crosslinked hydrophilic polymer, e.g., a covalently crosslinked hydrophilic polymer, a blend of a hydrophilic polymer and a relatively low molecular weight complementary oligomer that is capable of crosslinking the hydrophilic polymer via hydrogen bonding, or a combination thereof.

In a further embodiment, a tooth whitening composition is provided that comprises an admixture of:

a tooth whitening agent that is inert in a dry environment but activated in the presence of moisture; and

at least two water-swellable, water-insoluble polymers, wherein a first water-swellable, water-insoluble polymer is cationic, a second water-swellable, water-insoluble polymer is anionic, and the polymers are ionically associated with each other to form a polymer matrix.

In this embodiment, an ionically associated polymer matrix is provided as described above, but the composition contains a single tooth whitening agent that is moisture-activated. These compositions will also contain, in most instances, a crosslinked hydrophilic polymer as described above.

In another embodiment, a tooth whitening composition is provided that comprises: 1.5 wt. % to 30 wt. % of a hydrophilic polymer composition composed of (a) a covalently crosslinked hydrophilic polymer, and/or (b) a blend of a hydrophilic polymer and a complementary oligomer capable of hydrogen bonding thereto; 40 wt. % to 90 wt. % of at least one water-swellable, water-insoluble polymer; and at least one tooth whitening agent.

In another embodiment, a tooth whitening system is provided that comprises a flexible strip, or backing layer (also referred to herein as an “outer layer”), in contact with a tooth whitening composition of the invention. The backing layer may comprise any suitable material, e.g., polymer, woven, non-woven, foil, paper, rubber, or a combination thereof, such as a laminate. The backing layer may be erodible, as described in U.S. Patent Publication No. 2004/0105834. Generally, the system will also include a removal release liner that covers the tooth whitening composition prior to use and prevents exposure of the composition to air.

In a further embodiment, a tooth whitening system is provided in the form of a flexible, laminated tooth whitening strip that comprises:

a permeable outer layer that provides the outer surface of the strip following application to the teeth, the outer layer comprised of a relatively hydrophobic polymer and containing 1.0 to 10.0 wt. % of at least one tooth whitening agent; and

an interior whitening agent layer composed of a polymeric matrix containing 1.0 to 50.0 wt. % of at least one tooth whitening agent, the interior layer capable of adhering to the teeth in the presence of moisture.

In this embodiment, the system includes two flexible, soft layers with differential permeability, the outer layer being measurably permeable but somewhat less permeable than the inner layer. Tooth whitening agent is present in both layers, with the outer layer essentially serving as an additional reservoir for the whitening agent(s). The outer layer is relatively hydrophobic, such that the system is prevented from sticking to the lips and releasing any significant amount of hydrogen peroxide into the mouth in a direction away from the teeth.

### BRIEF DESCRIPTION OF THE FIGURES

FIG. 1 schematically illustrates a representative tooth whitening system of the invention in the form of a laminated adhesive strip.

FIG. 2 is a graph illustrating the flux (in mg/cm²/min) of hydrogen peroxide released in vitro for the systems evaluated in Example 3.

FIG. 3 is a graph illustrating the cumulative amount of hydrogen peroxide released in vitro for the systems evaluated in Example 3.

FIG. 4 is a graph illustrating the flux (in mg/cm²/min) of hydrogen peroxide released in vitro for the systems evaluated in Example 4.

FIG. 5 is a graph illustrating the cumulative amount of hydrogen peroxide released in vitro for the systems evaluated in Example 4.

FIG. 6 is a graph illustrating the flux (in mg/cm²/min) of hydrogen peroxide released in vivo for the systems evaluated in Example 5.

FIG. 7 is a graph illustrating the cumulative amount of hydrogen peroxide released in vivo for the systems evaluated in Example 5.

### DETAILED DESCRIPTION OF THE INVENTION

Before describing the present invention in detail, it is to be understood that unless otherwise indicated this invention is not limited to specific formulation materials or manufacturing processes, as such may vary. It is also to be understood that the terminology used herein is for the purpose of describing particular embodiments only, and is not intended to be limiting. It must be noted that, as used in this specification and the appended claims, the singular forms “a,” “an,” and “the” include plural referents unless the context clearly dictates otherwise. Thus, for example, reference to “a hydrophilic polymer” includes not only a single hydrophilic polymer but also a combination or mixture of two or more different hydrophilic polymers, reference to “a plasticizer” includes a combination or mixture of two or more different plasticizers as well as a single plasticizer, and the like.

In describing and claiming the present invention, the following terminology will be used in accordance with the definitions set out below.

The definitions of “hydrophobic” and “hydrophilic” polymers are based on the amount of water vapor absorbed by polymers at 100% relative humidity. According to this classification, hydrophobic polymers absorb only up to 1 wt. % water at 100% relative humidity (“rh”), while moderately hydrophilic polymers absorb 1-10 wt. % water, hydrophilic polymers are capable of absorbing more than 10 wt. % of water, and hygroscopic polymers absorb more than 20 wt. % of water. A “water-swellable” polymer is one that absorbs an amount of water greater than at least 50 wt. % of its own weight, upon immersion in an aqueous medium.

The term “crosslinked” herein refers to a composition containing intramolecular and/or intermolecular crosslinks, whether arising through covalent or noncovalent bonding. “Noncovalent” bonding includes both hydrogen bonding and electrostatic (ionic) bonding.

The term “polymer” includes linear and branched polymer structures, and also encompasses crosslinked polymers as well as copolymers (which may or may not be crosslinked), thus including block copolymers, alternating copolymers, random copolymers, and the like. Those compounds referred to herein as “oligomers” are polymers having a molecular weight below about 1000 Da, preferably below about 800 Da.

In a first embodiment, a tooth whitening formulation is provided that comprises a first tooth whitening agent that is inert in a dry environment but activated in the presence of moisture to release peroxide and produce an alkaline pH, a second tooth whitening agent that releases peroxide rapidly upon contact with moisture in the presence of base, and at least one water-swellable, water-insoluble polymer. The first tooth whitening agent may be, for example, an addition compound of (a) a salt of an oxyanion and (b) hydrogen peroxide. Such tooth whitening agents include, without limitation, sodium percarbonate (2Na₂CO₃.3H₂O₂; also known as sodium carbonate peroxyhydrate and peroxy sodium carbonate), which breaks down to sodium carbonate and hydrogen peroxide in water, with a resultant increase in the pH of the solution. Such tooth whitening agents also include sodium perborate (NaBO₃), sodium perborate monohydrate, and sodium perborate tetrahydrate. The second tooth whitening agent may be, for example, carbamide peroxide (CO(NH₂)₂H₂O₂; also known as urea peroxide), or selected from any number of other organic and inorganic compounds that release peroxide rapidly in the presence of aqueous base.

The water-swellable, water-insoluble polymer is capable of at least some degree of swelling when immersed in an aqueous liquid but is either completely insoluble in water or water-insoluble within a selected pH range, generally up to a pH of at least about 7.5 to 8.5. The polymer may be comprised of a cellulose ester, for example, cellulose acetate, cellulose acetate propionate (CAP), cellulose acetate butyrate (CAB), cellulose propionate (CP), cellulose butyrate (CB), cellulose propionate butyrate (CPB), cellulose diacetate (CDA), cellulose triacetate (CTA), or the like. Cellulose esters are described in U.S. Pat. Nos. 1,698,049, 1,683,347, 1,880,808, 1,880,560, 1,984,147, 2,129,052, and 3,617,201, and may be prepared using techniques known in the art or obtained commercially. Commercially available cellulose esters suitable herein include CA 320, CA 398, CAB 381, CAB 551, CAB 553, CAP 482, CAP 504, all available from Eastman Chemical Company, Kingsport, Term. Such cellulose esters typically have a number average molecular weight of between about 10,000 and about 75,000.

Generally, cellulose esters comprise a mixture of cellulose and cellulose ester monomer units; for example, commercially available cellulose acetate butyrate contains cellulose acetate monomer units as well as cellulose butyrate monomer units and unesterified cellulose units. Preferred cellulose esters herein are cellulose acetate butyrate compositions and cellulose acetate propionate compositions with the following properties: cellulose acetate butyrate, butyrate content 17-52%, acetyl content 2.0-29.5%, unesterified hydroxyl content, 1.1-4.8%, molecular weight 12,000-20,000 g/mole, glass transition temperature T_{g} in the range of 96-141° C., and melting temperature in the range of 130-240° C.; and cellulose acetate propionate, propionate content 42.5-47.7%, acetyl content 0.6-1.5%, unesterified hydroxyl content, 1.7-5.0%, molecular weight 15,000-75,000 g/mole, glass transition temperature T_{g} in the range of 142-159° C., and melting temperature in the range of 188-210° C. Suitable cellulosic polymers typically have an inherent viscosity (I.V.) of about 0.2 to about 3.0 deciliters/gram, preferably about 1 to about 1.6 deciliters/gram, as measured at a temperature of 25° C. for a 0.5 gram sample in 100 ml of a 60/40 by weight solution of phenol/tetrachloroethane.

Other preferred water-swellable polymers are acrylate polymers, generally formed from acrylic acid, methacrylic acid, acrylate, methyl acrylate, ethyl acrylate, methyl methacrylate, ethyl methacrylate, a dialkylaminoalkyl acrylate, a dialkylaminoalkyl methacrylate, a trialkylammonioalkyl acrylate, and/or a trialkylammonioalkyl methacrylate. Preferred such polymers are copolymers of acrylic acid, methacrylic acid, methyl methacrylate, ethyl methacrylate, 2-dimethylaminoethyl methacrylate, and/or trimethylammonioethyl methacrylate chloride.

Suitable acrylate polymers are those copolymers available under the tradename “Eudragit” from Rohm Pharma (Germany). The Eudragit series E, L, S, RL, RS and NE copolymers are available as solubilized in organic solvent, in an aqueous dispersion, or as a dry powder. Preferred acrylate polymers are copolymers of methacrylic acid and methyl methacrylate, such as the Eudragit L and Eudragit S series polymers. Particularly preferred such copolymers are Eudragit L-30D-55 and Eudragit L-100-55 (the latter copolymer is a spray-dried form of Eudragit L-30D-55 that can be reconstituted with water). The molecular weight of the Eudragit L-30D-55 and Eudragit L-100-55 copolymer is approximately 135,000 Da, with a ratio of free carboxyl groups to ester groups of approximately 1:1. The copolymer is generally insoluble in aqueous fluids having a pH below 5.5. Another particularly suitable methacrylic acid-methyl methacrylate copolymer is Eudragit S-100, which differs from Eudragit L-30D-55 in that the ratio of free carboxyl groups to ester groups is approximately 1:2. Eudragit S-100 is insoluble at pH below 5.5, but unlike Eudragit L-30D-55, is poorly soluble in aqueous fluids having a pH in the range of 5.5 to 7.0. This copolymer is soluble at pH 7.0 and above. Eudragit L-100 may also be used, which has a pH-dependent solubility profile between that of Eudragit L-30D-55 and Eudragit S-100, insofar as it is insoluble at a pH below 6.0. It will be appreciated by those skilled in the art that Eudragit L-30D-55, L-100-55, L-100, and S-100 can be replaced with other acceptable polymers having similar pH-dependent solubility characteristics.

Other preferred acrylate polymers are cationic, such as the Eudragit E, RS, and RL series polymers. Eudragit E100 and E PO are cationic copolymers of dimethylaminoethyl methacrylate and neutral methacrylates (e.g., methyl methacrylate), while Eudragit RS and Eudragit RL polymers are analogous polymers, composed of neutral methacrylic acid esters and a small proportion of trimethylammonioethyl methacrylate.

In this embodiment, the formulation may contain a single water-swellable, water-insoluble polymer as described above. Alternatively, an admixture of at least two water-swellable, water-insoluble polymers may be present. In the latter case, an exemplary formulation is provided by combining a cationic water-swellable, water-insoluble polymer with an anionic water swellable, water-insoluble polymer, such that the polymers are ionically associated with each other and form a polymer matrix. For example, the cationic polymer may be an acrylate-based polymer with pendant quaternary ammonium groups or tertiary amino groups (as exemplified by a Eudragit RS, Eudragit RL, Eudragit E copolymer), and the anionic polymer may be an ionized acrylic acid or methacrylic acid polymer such as a Eudragit L or Eudragit S copolymer. The anionic polymer may also be, for example, hydroxypropyl methylcellulose phthalate.

The tooth whitening formulation will generally include a crosslinked hydrophilic polymer as well. The crosslinked hydrophilic polymer may be covalently crosslinked, ionically crosslinked, or crosslinked via hydrogen bonding, wherein crosslinking may be either intramolecular or intermolecular, and the formulations may contain any combinations of such crosslinked polymers. The hydrophilic polymer may be crosslinked via a crosslinking agent, e.g., via a low molecular weight complementary oligomer.

Suitable hydrophilic polymers include repeating units derived from an N-vinyl lactam monomer, a carboxy vinyl monomer, a vinyl ester monomer, an ester of a carboxy vinyl monomer, a vinyl amide monomer, and/or a hydroxy vinyl monomer. Such polymers include, by way of example, poly(N-vinyl lactams), poly(N-vinyl acrylamides), poly(N-alkylacrylamides), substituted and unsubstituted acrylic and methacrylic acid polymers, polyvinyl alcohol (PVA), polyvinylamine, copolymers thereof and copolymers with other types of hydrophilic monomers (e.g. vinyl acetate). Other suitable hydrophilic polymers include, but are not limited to: polysaccharides; crosslinked acrylate polymers and copolymers; carbomers, i.e., hydroxylated vinylic polymers (also referred to as “interpolymers”) which are prepared by crosslinking a monoolefinic acrylic acid monomer with a polyalkyl ether of sucrose (commercially available under the trademark Carbopol® from the B.F. Goodrich Chemical Company); crosslinked acrylamide-sodium acrylate copolymers; gelatin; vegetable polysaccharides, such as alginates, pectins, carrageenans, or xanthan; starch and starch derivatives; and galactomannan and galactomannan derivatives.

Polysaccharide materials include, for instance, crosslinked, normally water-soluble cellulose derivatives that are crosslinked to provide water-insoluble, water-swellable compounds, such as crosslinked sodium carboxymethylcellulose (CMC), crosslinked hydroxyethyl cellulose (HEC), crosslinked partial free acid CMC, and guar gum grafted with acrylamide and acrylic acid salts in combination with divinyl compounds, e.g., methylene-bis acrylamide. Within the aforementioned class, the more preferred materials are crosslinked CMC derivatives, particularly crosslinked sodium CMC and crosslinked HEC. Other polysaccharides suitable herein include hydroxypropyl cellulose (HPC), hydroxypropyl methylcellulose (HPMC), hydroxypropyl cellulose (HPC), and the like.

Poly(N-vinyl lactams) useful herein are preferably homopolymers or copolymers of N-vinyl lactam monomer units, with N-vinyl lactam monomer units representing the majority of the total monomeric units of a poly(N-vinyl lactams) copolymer. Preferred poly(N-vinyl lactams) for use in conjunction with the invention are prepared by polymerization of one or more of the following N-vinyl lactam monomers: N-vinyl-2-pyrrolidone; N-vinyl-2-valerolactam; and N-vinyl-2-caprolactam. Nonlimiting examples of non-N-vinyl lactam comonomers useful with N-vinyl lactam monomeric units include N,N-dimethylacrylamide, acrylic acid, methacrylic acid, hydroxyethylmethacrylate, acrylamide, 2-acrylamido-2-methyl-1-propane sulfonic acid or its salt, and vinyl acetate.

Poly (N-alkylacrylamides) include, by way of example, poly(methacrylamide) and poly(N-isopropyl acrylamide) (PNIPAM). Polymers of carboxy vinyl monomers are typically formed from acrylic acid, methacrylic acid, crotonic acid, isocrotonic acid, itaconic acid and anhydride, a 1,2-dicarboxylic acid such as maleic acid or fumaric acid, maleic anhydride, or mixtures thereof, with preferred hydrophilic polymers within this class including polyacrylic acid and polymethacrylic acid, with polyacrylic acid most preferred.

Preferred hydrophilic polymers herein are the following: poly(N-vinyl lactams), particularly polyvinyl pyrrolidone (PVP) and poly(N-vinyl caprolactam) (PVCap); poly(N-vinyl acetamides), particularly polyacetamide per se; polymers of carboxy vinyl monomers, particularly polyacrylic acid and polymethacrylic acid; and copolymers and blends thereof. PVP and PVCap are particularly preferred.

The molecular weight of the hydrophilic polymer is not critical; however, the number average molecular weight of the hydrophilic polymer is generally in the range of approximately 20,000 to 2,000,000, more typically in the range of approximately 200,000 to 1,000,000.

Covalent crosslinking may be accomplished in several ways. For instance, the hydrophilic polymer, or the hydrophilic polymer and a complementary oligomer, may be covalently crosslinked using heat, radiation, or a chemical curing (crosslinking) agent. Covalently crosslinked hydrophilic polymers may also be obtained commercially, for example, crosslinked sodium CMC is available under the tradename Aquasorb® (e.g., Aquasorb® A500) from Aqualon, a division of Hercules, Inc., and crosslinked PVP is available under the tradename Kollidon® (e.g., Kollidon® CL, and Kollidon® CL-M, a micronized form of crosslinked PVP, both available from BASF).

For thermal crosslinking, a free radical polymerization initiator is used, and can be any of the known free radical-generating initiators conventionally used in vinyl polymerization. Preferred initiators are organic peroxides and azo compounds, generally used in an amount from about 0.01 wt. % to 15 wt. %, preferably 0.05 wt. % to 10 wt. %, more preferably from about 0.1 wt. % to about 5% and most preferably from about 0.5 wt. % to about 4 wt. % of the polymerizable material. Suitable organic peroxides include dialkyl peroxides such as t-butyl peroxide and 2,2 bis(t-butylperoxy)propane, diacyl peroxides such as benzoyl peroxide and acetyl peroxide, peresters such as t-butyl perbenzoate and t-butyl per-2-ethylhexanoate, perdicarbonates such as dicetyl peroxy dicarbonate and dicyclohexyl peroxy dicarbonate, ketone peroxides such as cyclohexanone peroxide and methylethylketone peroxide, and hydroperoxides such as cumene hydroperoxide and tert-butyl hydroperoxide. Suitable azo compounds include azo bis(isobutyronitrile) and azo bis (2,4-dimethylvaleronitrile). The temperature for thermal crosslinking will depend on the actual components and may be readily determined by one of ordinary skill in the art, but typically ranges from about 80° C. to about 200° C.

Crosslinking may also be accomplished with radiation, typically in the presence of a photoinitator. The radiation may be ultraviolet, alpha, beta, gamma, electron beam, and x-ray radiation, although ultraviolet radiation is preferred. Useful photosensitizers are triplet sensitizers of the “hydrogen abstraction” type, and include benzophenone and substituted benzophenone and acetophenones such as benzyl dimethyl ketal, 4-acryloxybenzophenone (ABP), 1-hydroxy-cyclohexyl phenyl ketone, 2,2-diethoxyacetophenone and 2,2-dimethoxy-2-phenylaceto-phenone, substituted alpha-ketols such as 2-methyl-2-hydroxypropiophenone, benzoin ethers such as benzoin methyl ether and benzoin isopropyl ether, substituted benzoin ethers such as anisoin methyl ether, aromatic sulfonyl chlorides such as 2-naphthalene sulfonyl chloride, photoactive oximes such as 1-phenyl-1,2-propanedione-2-(O-ethoxy-carbonyl)-oxime, thioxanthones including alkyl- and halogen-substituted thioxanthonse such as 2-isopropylthioxanthone, 2-chlorothioxanthone, 2,4 dimethyl thioxanone, 2,4 dichlorothioxanone, and 2,4-diethyl thioxanone, and acyl phosphine oxides. Radiation having a wavelength of 200 to 800 nm, preferably, 200 to 500 nm, is preferred for use herein, and low intensity ultraviolet light is sufficient to induce crosslinking in most cases. However, with photosensitizers of the hydrogen abstraction type, higher intensity UV exposure may be necessary to achieve sufficient crosslinking. Such exposure can be provided by a mercury lamp processor such as those available from PPG, Fusion, Xenon, and others. Crosslinking may also be induced by irradiating with gamma radiation or an electron beam. Appropriate irradiation parameters, i.e., the type and dose of radiation used to effect crosslinking, will be apparent to those skilled in the art.

Suitable chemical curing agents, also referred to as chemical cross-linking “promoters,” include, without limitation, polymercaptans such as 2,2-dimercapto diethylether, dipentaerythritol hexa(3-mercaptopropionate), ethylene bis(3-mercaptoacetate), pentaerythritol tetra(3-mercaptopropionate), pentaerythritol tetrathioglycolate, polyethylene glycol dimercaptoacetate, polyethylene glycol di(3-mercaptopropionate), trimethylolethane tri(3-mercaptopropionate), trimethylolethane trithioglycolate, trimethylolpropane tri(3-mercapto-propionate), trimethylolpropane trithioglycolate, dithioethane, di- or trithiopropane and 1,6-hexane dithiol. The crosslinking promoter is added to the uncrosslinked hydrophilic polymer to promote covalent crosslinking thereof, or to a blend of the uncrosslinked hydrophilic polymer and the complementary oligomer, to provide crosslinking between the two components.

The crosslinked hydrophilic polymer may also comprise a blend of a hydrophilic polymer and a low molecular weight complementary oligomer capable of crosslinking the polymer via hydrogen bonding. In this case, the hydrophilic polymer may or may not be crosslinked prior to admixture with the complementary oligomer. If the hydrophilic polymer is crosslinked prior to admixture with the complementary oligomer, it may be preferred to synthesize the polymer in crosslinked form, by admixing a monomeric precursor to the polymer with multifunctional comonomer and copolymerizing. Examples of monomeric precursors and corresponding polymeric products are as follows: N-vinyl amide precursors for a poly(N-vinyl amide) product; N-alkylacrylamides for a poly(N-alkylacrylamide) product; acrylic acid for a polyacrylic acid product; methacrylic acid for a polymethacrylic acid product; acrylonitrile for a poly(acrylonitrile) product; and N-vinyl pyrrolidone (NVP) for a poly(vinylpyrrolidone) (PVP) product. Polymerization may be carried out in bulk, in suspension, in solution, or in an emulsion. Solution polymerization is preferred, and polar organic solvents such as ethyl acetate and lower alkanols (e.g., ethanol, isopropyl alcohol, etc.) are particularly preferred. For preparation of hydrophilic vinyl polymers, synthesis will typically take place via a free radical polymerization process in the presence of a free radical initiator as described above. The multifunctional comonomer include, for example, bisacrylamide, acrylic or methacrylic esters of diols such as butanediol and hexanediol (1,6-hexane diol diacrylate is preferred), other acrylates such as pentaerythritol tetraacrylate, and 1,2-ethylene glycol diacrylate, and 1,12-dodecanediol diacrylate. Other useful multifunctional crosslinking monomers include oligomeric and polymeric multifunctional (meth)acrylates, e.g., poly(ethylene oxide) diacrylate or poly(ethylene oxide) dimethacrylate; polyvinylic crosslinking agents such as substituted and unsubstituted divinylbenzene; and difunctional urethane acrylates such as EBECRYL® 270 and EBECRYL® 230 (1500 weight average molecular weight and 5000 weight average molecular weight acrylated urethanes, respectively—both available from UCB of Smyrna, Ga.), and combinations thereof. If a chemical crosslinking agent is employed, the amount used will preferably be such that the weight ratio of crosslinking agent to hydrophilic polymer is in the range of about 1:100 to 1:5. To achieve a higher crosslink density, if desired, chemical crosslinking is combined with radiation curing.

If the crosslinked hydrophilic polymer is in the form of a blend of a hydrophilic polymer and a low molecular weight complementary oligomer, the blend will usually provide a matrix that is crosslinked solely by hydrogen bonds formed between the termini of the oligomer and pendant groups on the hydrophilic polymer. In this embodiment, suitable hydrophilic polymers include repeating units derived from an N-vinyl lactam monomer, a carboxy vinyl monomer, a vinyl ester monomer, an ester of a carboxy vinyl monomer, a vinyl amide monomer, and/or a hydroxy vinyl monomer, as described above with regard to crosslinked hydrophilic polymers per se, and preferred hydrophilic polymers in this blend are also as described above for those polymers.

The oligomer is generally “complementary” to the hydrophilic polymers in that it is capable of hydrogen bonding thereto. Preferably, the complementary oligomer is terminated with hydroxyl groups, amino or carboxyl groups. The oligomer typically has a glass transition temperature T_{g} in the range of about −100° C. to about −30° C. and a melting temperature Tₘ lower than about 20° C. The oligomer may be also amorphous. The difference between the T_{g} values the hydrophilic polymer and the oligomer is preferably greater than about 50° C., more preferably greater than about 100° C., and most preferably in the range of about 150° C. to about 300° C. The hydrophilic polymer and complementary oligomer should be compatible, i.e. capable of forming a homogeneous blend that exhibits a single T_{g}, intermediate between those of the unblended components. Generally, the oligomer will have a molecular weight in the range from about 45 to about 800, preferably in the range of about 45 to about 600. Examples of suitable oligomers include, but are not limited to, low molecular weight polyalcohols (e.g. glycerol), oligoalkylene glycols such as ethylene glycol and propylene glycol, ether alcohols (e.g., glycol ethers), alkane diols from butane diol to octane diol, and carboxyl-terminated and amino-terminated derivatives of polyalkylene glycols. Polyalkylene glycols, optionally carboxyl-terminated, are preferred herein, and polyethylene glycol having a molecular weight in the range of about 300 to 600 is an optimal complementary oligomer.

The hydrophilic polymer and the complementary oligomer should be miscible with respect to each other and have disparate chain lengths (as may be deduced from the above). The ratio of the weight average molecular weight of the hydrophilic polymer to that of the oligomer should be within about 200 and 200,000, preferably within about 1,250 and 20,000. Also, the polymer and the oligomer should contain complementary functional groups capable of hydrogen bonding, ionically bonding, or covalently bonding to each other. Ideally, the complementary functional groups of the polymer are located throughout the polymeric structure, while the functional groups of the oligomer are preferably located at the two termini of a linear molecule, and are not present along the backbone. Forming hydrogen bonds or ionic bonds between the two terminal functional groups of the oligomer and the corresponding functional groups contained along the backbone of the hydrophilic polymer results in a noncovalently linked supramolecular network.

As discussed in U.S. Pat. No. 6,576,712 to Feldstein et al., the ratio of the hydrophilic polymer to the complementary oligomer in the aforementioned blend affects both adhesive strength and cohesive strength. As explained in the aforementioned patent, the complementary oligomer decreases the glass transition of the hydrophilic polymer/complementary oligomer blend to a greater degree than predicted by the Fox equation, which is given by equation (1)

$$\frac{1}{{T}_{g predicted}} = \frac{{w}_{pol}}{{T}_{g pol}} + \frac{{w}_{pl}}{{T}_{g pl}} \qquad ( 1 )$$

where T_{g predicted} is the predicted glass transition temperature of the hydrophilic polymer/complementary oligomer blend, wₚₒₗ is the weight fraction of the hydrophilic polymer in the blend, wₚₗ is the weight fraction of the complementary oligomer in the blend, T_{g pol} is the glass transition temperature of the hydrophilic polymer, and T_{g pl} is the glass transition temperature of the complementary oligomer. As also explained in that patent, an adhesive composition having optimized adhesive and cohesive strength can be prepared from a hydrophilic polymer and a complementary oligomer by selecting the components and their relative amounts to give a predetermined deviation from T_{g predicted}. Generally, to maximize adhesion, the predetermined deviation from T_{g predicted} will be the maximum negative deviation, while to minimize adhesion, any negative deviation from T_{g predicted} is minimized. Optimally, the complementary oligomer represents approximately 25 wt. % to 75 wt. %, preferably about 30 wt. % to about 60 wt. %, of the hydrophilic polymer/complementary oligomer blend, and, correspondingly, the hydrophilic polymer represents approximately 75 wt. % to 25 wt. %, preferably about 70 wt. % to about 40 wt. %, of the hydrophilic polymer/oligomer blend.

For certain applications, particularly when a relatively high cohesive strength formulation is desired, the hydrophilic polymer, and optionally the complementary oligomer should be covalently crosslinked. The hydrophilic polymer may be covalently crosslinked, either intramolecularly or intermolecularly, and/or the hydrophilic polymer and the complementary oligomer may be covalently crosslinked. In the former case, there are no covalent bonds linking the hydrophilic polymer to the complementary oligomer, while in the latter case, there are covalent crosslinks binding the hydrophilic polymer to the complementary oligomer. The hydrophilic polymer, or the hydrophilic polymer and the complementary oligomer, may be covalently crosslinked using heat, radiation, or a chemical curing (crosslinking) agent. The degree of crosslinking should be sufficient to eliminate or at least minimize cold flow under compression.

For covalently crosslinked hydrophilic polymer/complementary oligomer systems, the oligomer should be terminated at each end with a group capable of undergoing reaction with a functional group on the hydrophilic polymer. Such reactive groups include, for example, hydroxyl groups, amino groups, and carboxyl groups. These difunctionalized oligomers may be obtained commercially or readily synthesized using techniques known to those of ordinary skill in the art and/or described in the pertinent texts and literature.

As the complementary oligomer may itself act as a plasticizer, it is not generally necessary to incorporate an added low molecular weight plasticizer into the present compositions unless the optional complementary oligomer is not included. Suitable low molecular weight plasticizers include: dialkyl phthalates, dicycloalkyl phthalates, diaryl phthalates, and mixed alkyl-aryl phthalates, as represented by dimethyl phthalate, diethyl phthalate, dipropyl phthalate, di(2-ethylhexyl)-phthalate, di-isopropyl phthalate, diamyl phthalate and dicapryl phthalate; alkyl and aryl phosphates such as tributyl phosphate, trioctyl phosphate, tricresyl phosphate, and triphenyl phosphate; alkyl citrate and citrate esters such as trimethyl citrate, triethyl citrate, tributyl citrate, acetyl triethyl citrate, and trihexyl citrate; dialkyl adipates such as dioctyl adipate (DOA; also referred to as bis(2-ethylhexyl)adipate), diethyl adipate, di(2-methylethyl)adipate, and dihexyl adipate; dialkyl tartrates such as diethyl tartrate and dibutyl tartrate; dialkyl sebacates such as diethyl sebacate, dipropyl sebacate and dinonyl sebacate; dialkyl succinates such as diethyl succinate and dibutyl succinate; alkyl glycolates, alkyl glycerolates, glycol esters and glycerol esters such as glycerol diacetate, glycerol triacetate (triacetin), glycerol monolactate diacetate, methyl phthalyl ethyl glycolate, butyl phthalyl butyl glycolate, ethylene glycol diacetate, ethylene glycol dibutyrate, triethylene glycol diacetate, triethylene glycol dibutyrate and triethylene glycol dipropionate; and mixtures thereof. Preferred low molecular weight plasticizers for the continuous hydrophilic phase are triethyl citrate, diethyl phthalate, and dioctyl adipate, with dioctyl adipate most preferred.

The properties of the compositions of the invention are readily controlled by adjusting one or more parameters during formulation. For example, the adhesiveness of the composition can be controlled during manufacture in order to increase or decrease the degree to which the composition will adhere to the teeth in the presence of moisture. This can be accomplished by varying type and/or amount of different components, or by changing the mode of manufacture. Also, with respect to the fabrication process, compositions prepared using a conventional melt extrusion process are generally, although not necessarily, somewhat less tacky than compositions prepared using a solution cast technique.

In another embodiment, a tooth whitening composition is provided that is composed of an admixture of a tooth whitening agent, generally, although not necessarily, one that is inert in a dry environment but activated in the presence of moisture, and at least two water-swellable, water-insoluble polymers, wherein a first water-swellable, water-insoluble polymer is cationic, a second water-swellable, water-insoluble polymer is anionic, and the polymers are ionically associated with each other to form a polymer matrix. In this embodiment, the composition may contain a single tooth whitening agent, but necessarily includes a mixture of ionically associated polymers as are present in the preferred embodiment discussed above. The cationic polymer may be, for example, an acrylate-based polymer with pendant quaternary ammonium groups, and the anionic polymer may be an ionized acrylic acid or methacrylic acid polymer. Specific such polymers are as described earlier herein.

In an additional embodiment, a tooth whitening composition is provided that is composed of an admixture of: 1.5 wt. % to 30 wt. %, preferably 1.5 wt. % to 20 wt. %, more preferably 1.5 wt. % to 90 wt. %, and most preferably 1.5 wt. % to 95 wt. %, of a hydrophilic polymer composition composed of (a) a covalently crosslinked hydrophilic polymer, and/or (b) a blend of a hydrophilic polymer and a complementary oligomer capable of hydrogen bonding thereto; 40 wt. % to 90 wt. %, preferably 45 wt. % to 90 wt. %, more preferably 50 wt. % to 90 wt. %, and most preferably 60 wt. % to 90 wt. %, of at least one water-swellable, water-insoluble polymer; and at least one tooth whitening agent.

In these embodiments, suitable tooth whitening agents include peroxides, metal chlorites (e.g., calcium chlorite and sodium chlorite), perborates (e.g., sodium perborate), percarbonates (e.g., sodium percarbonate), peroxyacids (e.g., diperoxydodecanoic acid), and combinations thereof. Peroxides are preferred; representative peroxides include hydrogen peroxide, calcium peroxide, carbamide peroxide, dialkyl peroxides such as t-butyl peroxide and 2,2 bis(t-butylperoxy)propane, diacyl peroxides such as benzoyl peroxide and acetyl peroxide, peresters such as t-butyl perbenzoate and t-butyl per-2-ethylhexanoate, perdicarbonates such as dicetyl peroxy dicarbonate and dicyclohexyl peroxy dicarbonate, ketone peroxides such as cyclohexanone peroxide and methylethylketone peroxide, and hydroperoxides such as cumene hydroperoxide and tert-butyl hydroperoxide.

The tooth whitening compositions of the invention may include any of a number of optional additives, such as anti-tartar agents, enzymes, flavoring agents, sweeteners, fillers, preservatives, and breath fresheners.

Anti-tartar agents include phosphates such as pyrophosphates, polyphosphates, polyphosphonates (e.g., ethane-1-hydroxy-1,1-diphosphonate, 1-azacycloheptane-1,1-diphosphonate, and linear alkyl diphosphonates), and salts thereof; linear carboxylic acids; and sodium zinc citrate; and mixtures thereof. Preferred pyrophosphate salts are the alkali metal pyrophosphate salts and the hydrated or unhydrated forms of disodium dihydrogen pyrophosphate (Na₂H₂P₂O₇), tetrasodium pyrophosphate (Na₄P₂O₇), and tetrapotassium pyrophosphate (K₄P₂O₇). Anti-tartar agents also include betaines and amine oxides, as described in U.S. Pat. No. 6,315,991 to Zofchak.

Enzymes useful in inhibiting the formation of plaque, calculus, or dental caries are also useful in the compositions. Such enzymes include: proteases that break down salivary proteins which are absorbed onto the tooth surface and form the pellicle, or first layer of plaque; lipases which destroy bacteria by lysing proteins and lipids which form the structural component of bacterial cell walls and membranes; dextranases, glucanohydrolases, endoglycosidases, and mucinases which break down the bacterial skeletal structure which forms a matrix for bacterial adhesion to the tooth; and amylases which prevent the development of calculus by breaking-up the carbohydrate-protein complex that binds calcium. Preferred enzymes include any of the commercially available proteases; dextranases; glucanohydrolases; endoglycosidases; amylases; mutanases; lipases; mucinases; and compatible mixtures thereof.

Any natural or synthetic flavorants can be used. Suitable flavorants include wintergreen, peppermint, spearmint, menthol, fruit flavors, vanilla, cinnamon, spices, flavor oils, and oleoresins, as known in the art, as well as combinations thereof. The amount of flavorant employed is normally a matter of preference, subject to such factors as flavor type, individual flavor, and strength desired. Preferably, the composition comprises from about 0.1 wt % to about 5 wt % flavorant. Sweeteners useful in the present compositions include sucrose, fructose, aspartame, xylitol and saccharine.

The compositions may also contain active agents for treating adverse conditions of the teeth and surrounding tissue, e.g., periodontal and oral infections, periodontal lesions, dental caries or decay, and gingivitis. The active agent can be, for example, a non-steroidal anti-inflammatory/analgesic, a steroidal anti-inflammatory agents, a local anesthetic agent, a bactericidal agent, an antibiotic, an antifungal agent, or a tooth desensitizing agent. See, e.g., U.S. Patent Publication No. US 2003/0152528 A1 to Singh et al., published Aug. 14, 2003, the disclosure of which is incorporated by reference herein.

The tooth whitening formulations of the invention are generally melt extrudable, and thus may be prepared using a simple blending and extruding process. The components of the composition are weighed out and then admixed, for example using a Brabender or Baker Perkins Blender, generally although not necessarily at an elevated temperature, e.g., about 90° C. to about 140° C. The resulting formulation can be extruded using a single or twin extruder, or pelletized. Preferably the formulation is extruded directly onto a substrate such as a backing layer or release liner, and then pressed. In a particularly preferred embodiment, the formulation is extruded onto an outer layer composed of a permeable polymer matrix, as described in Example 2. The thickness of the resulting laminate will be in the range of about 0.05 mm to about 0.80 mm, more usually in the range of about 0.1 mm to about 0.25 mm. Other manufacturing processes, e.g., solvent casting as described in No. US 2003/0152528 A1 to Singh et al., cited supra, can also be employed.

The tooth whitening compositions of the invention can be applied to the teeth in any suitable manner, although it is preferred that the compositions be present as a layer on a flexible strip of material that is applied across a row of teeth as a “tooth whitening strip.” In a further embodiment of the invention, then, a tooth whitening system is provided that comprises an outer backing layer that provides the external surface of the system following application to the teeth; a layer of a tooth whitening composition of the invention in contact therewith; and a removable release liner of polyethylene terephthalate (PET) or the like that covers the otherwise exposed tooth whitening composition prior to use. The backing layer is composed of an inert material, e.g., polyester, polyethylene, polypropylene, polyurethane, or the like. Ideally, the backing is relatively soft and flexible so as to permit the system to conform to the contour of the teeth and minimize any discomfort to the user.

An erodible backing layer may be used which is comprised of a polymer composition that erodes in a moist environment at a slower rate than the hydrogel and is substantially non-tacky. There are numerous materials that can be used for the backing member, and include, by way of example, and not limitation, acrylate polymers, cellulose derived polymers, cellulose esters, starches, alginic acid, alginates, polyamino acids. Combinations, i.e., blends of any of these different polymers can also serve as backing member material.

In one embodiment, the hydrogel erodes in about 1 second to 24 hours after placement in a moist environment, and in another embodiment the hydrogel erodes about 10seconds to 8 hours after placement. The erodible backing member, in one embodiment, erodes about 12 to 24 hours after the hydrogel has eroded, while in another embodiment the backing erodes within about 12 hours after hydrogel has eroded. The erodible backing member material can be selected so as to erode at a slightly slower or approximately the same rate (e.g., when they both erode within about 24 hours), but is preferably selected so that it erodes at a slower rate than the hydrogel composition, when in use. In one embodiment, the erodible backing member erodes at least about 200% slower than the hydrogel, in another embodiment, the backing erodes at least about 100% slower, in a different embodiment the backing erodes at least about 50% slower, and in yet another embodiment the backing erodes at least about 25% slower than the hydrogel.

Suitable acrylate polymers are described above as water-swellable, water-insoluble polymers, and include by way of example and not limitation, polymers formed from acrylic acid, methacrylic acid, methyl acrylate, ethyl acrylate, methyl methacrylate, ethyl methacrylate, and/or other vinyl monomers. Preferred acrylate polymers are the Eudragit® copolymers (copolymers of methacrylic acid and methyl methacrylate), such as the Eudragit® series E, L, S, RL, RS and NE copolymers. As noted above, these Eudragit polymers also find utility as the water-swellable, water-insoluble polymer component of the hydrogel. Since Eudragit polymers are available in different grades with varying pH dependent solubility and permeability characteristics, the grade used for the erodible backing can be selected to have a lower solubility as compared to the grade used in the hydrogel. For example, if L 100-55 is selected for use in the hydrogel, Eudragit L 100 can be used in the backing; if Eudragit L 100 is used in hydrogel, Eudragit S 100 could be used in the backing; and so forth. In addition, mixtures of Eudragit polymers or mixtures of Eudragit polymers with other polymers and excipients (e.g. buffering agents, pH modulators) may be used to tailor the rate of erosion of the backing member relative to the hydrogel.

Suitable cellulose derived polymers include by way of example and not limitation, hydratecellulose (cellophane), methyl cellulose, ethyl cellulose, hydroxyethyl cellulose (HEC), hydroxypropylcellulose (HPC), hydroxypropylmethylcellulose (HPMC), carboxymethylcellulose (CMC), and sodium carboxymethylcellulose (Na—CMC). Preferred celluloses are hydratecellulose, methyl cellulose, ethyl cellulose, hydroxyethyl cellulose, hydroxypropylcellulose, hydroxypropylmethylcellulose, carboxymethylcellulose, sodium carboxymethylcellulose, and mixtures thereof.

Suitable cellulose esters include by way of example and not limitation, cellulose acetate, cellulose acetate propionate, cellulose acetate butyrate, cellulose propionate, cellulose butyrate, cellulose propionate butyrate, cellulose diacetate, cellulose triacetate, and mixtures, polymers and copolymers thereof. Exemplary cellulose ester copolymers include cellulose acetate butyrate and cellulose acetate proprionate. Preferred cellulose esters are cellulose acetate, cellulose acetate propionate, cellulose acetate butyrate, cellulose propionate, cellulose butyrate, cellulose propionate butyrate, cellulose diacetate, cellulose triacetate, cellulose acetate butyrate, and cellulose acetate proprionate and mixtures thereof.

Suitable starches include by way of example and not limitation, potato starch acetate, maize starch, etc. (e.g., Clearam® starches sold by Roquette), and mixtures thereof.

Suitable alginates include by way of example and not limitation, propylene glycol alginate, sodium alginate, calcium alginate, and so forth, as well as mixtures thereof.

Suitable polyamino acids include by way of example and not limitation, polylysine, polyglycine, polyalanine, protamine, and so forth, as well as mixtures thereof.

It is understood that any of the whitening agents and other ingredients described in relation to the hydrogel composition can also be present in the backing member. For example, the hydrogel may contain an active agent that is released onto a tooth surface or oral mucosa, while the backing can be loaded with a flavorant, which is released to oral cavity.

In a still further embodiment of the invention, a tooth whitening system is provided in the form of a flexible, laminated strip in which a tooth whitening composition as described above, containing approximately 1.0 wt. % to 50.0 wt. %, preferably 1.0 wt. % to 30.0 wt. %, of at least one tooth whitening agent, serves as an “interior,” tooth-contacting layer, and a second layer, adjacent to the tooth-contacting layer and comprised of a hydrophobic polymer containing 1.0 wt. % to 30.0 wt. %, preferably 1.0 wt. % to 10 wt. %, of at least one tooth whitening agent, serves as the outer surface of the strip following application of the system to the teeth. The interior layer is capable of adhering to the teeth in the presence of moisture. In this embodiment, then, a tooth whitening system is provided that includes two flexible, soft layers with differential permeability, the outer layer being measurably permeable but somewhat less permeable than the inner layer. Tooth whitening agent is present in both layers, with the outer layer essentially serving as an additional reservoir for the whitening agent(s). The outer layer is relatively hydrophobic (i.e., hydrophobic relative to the polymer(s) of the interior layer) such that the system is prevented from sticking to the lips and releasing any significant amount of hydrogen peroxide into the mouth in a direction away from the teeth. The outer layer may also contain inert and/or active additives as described above with regard to the tooth whitening composition per se. A particularly preferred polymer suitable as the primary component of the outer layer is Eudragit® RS-PO, which, as noted earlier herein, is a copolymer of neutral methacrylic acid esters and a small proportion of trimethylammonioethyl methacrylate.

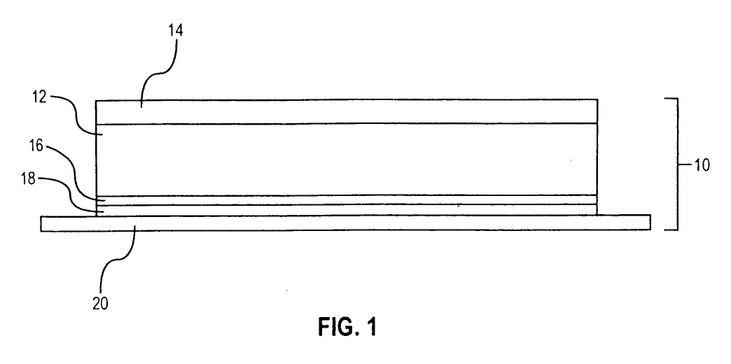

A representative tooth whitening system of the invention is illustrated schematically in FIG. 1. The system 10 is composed of an interior tooth whitening layer bisected by a nonwoven layer 16, such that the interior tooth whitening layer includes an upper region 12 and a lower region 18. The upper region is laminated to the outer backing layer 14, composed of a relatively hydrophobic, permeable polymer and containing 1.0 wt. % to 30.0 wt. % tooth whitening agent. Layer 14, as may be seen, provides the exterior surface of the system following application to the teeth. Removable release liner 20 covers the otherwise exposed surface of the lower region 18 of the interior tooth whitening layer prior to use.

The tooth whitening compositions of the invention are used by removing the product from its package, typically a moisture-free sealed pouch, removing the release liner, and applying the adhesive layer to the teeth. The tooth whitening systems described herein can be provided in a variety of sizes, so that the composition can be applied to the entirety or any portion of a tooth, and to any number of teeth at one time. The system can be left in place for an extended period of time, typically in the range of about 10 minutes to 8 hours, preferably in the range of about 30 to 60 minutes. The system can be readily removed by peeling it away from the surface of the teeth.

It is to be understood that while the invention has been described in conjunction with specific embodiments thereof, the foregoing description as well as the examples that follow are intended to illustrate and not limit the scope of the invention. Other aspects, advantages, and modifications within the scope of the invention will be apparent to those skilled in the art to which the invention pertains. All patents, patent applications, patent publications, journal articles, and other references cited herein are incorporated by reference in their entireties.

### EXPERIMENTAL

The following examples are put forth so as to provide those of ordinary skill in the art with a complete disclosure and description of how to make the tooth whitening formulations and systems of the invention. Efforts have been made to ensure accuracy with respect to numbers (e.g., amounts, temperature, etc.) but some experimental error and deviations should, of course, be allowed for. Unless indicated otherwise, parts are parts by weight, temperature is in degrees centigrade, and pressure is at or near atmospheric.

### Example 1

A tooth whitening formulation of the invention containing the following components was prepared using a hot melt processing method:

Eudragit RL PO or Eudragit E-PO, 19.00 g (38.00 wt. %)

Eudragit L100-55, 1.89 g (3.79 wt. %)

Triethyl citrate, 10.15 g (20.31 wt. %)

Kollidon CL-M, 7.50 g (15.00 wt. %)

Sodium percarbonate, 8.81 g (17.63 wt. %)

Carbamide peroxide, 2.64 g (5.28 wt. %)

The sodium percarbonate (obtained from Spectrum) was micronized using a Bell-art Products Micro Mill and sieved through an ASTM E-11 standard sieve #270 (53 microns; 0.0021″). The two Eudragit copolymers, the triethyl citrate (Morflex), and the Kollidon CL-M were weighed into a stainless steel tumbler and hand-mixed. The mixture was then transferred into a Brabender extruder, and mixed for 20 minutes at 80 rpm and a temperature of 130° C. Mixing speed was then adjusted to 5 rpm, and the carbamide peroxide and micronized sodium percarbonate were then added into the extruder. Mixing speed was then increased to 35-50 rpm and mixing was carried out for an additional 10 minutes at a temperature not exceeding 65° C.

### Example 2

A two-layer tooth whitening system of the invention, in which the tooth whitening formulation of Example 1 serves as the inner tooth whitening layer, was prepared as follows.

The outer layer of the tooth whitening system was prepared using the following components:

Eudragit RS-PO, 36.03 g (72.06 wt. %)

Triethyl citrate, 10.15 g (20.31 wt. %)

Sodium percarbonate, 2.94 g (5.88 wt. %)

Carbamide peroxide, 0.88 g (1.76 wt. %)

The Eudragit RS-PO and triethyl citrate were mixed by hand, and the mixture was then transferred into a Brabender extruder. The components were mixed for 20 minutes at 80 rpm and a temperature of 130° C. Mixing speed was then adjusted to 5 rpm, and the carbamide peroxide and sodium percarbonate (micronized as in Example 1) were then added into the extruder. Mixing speed was then increased to 35-50 rpm and mixing was carried out for an additional 10 minutes at a temperature not exceeding 65° C.

The mixture so provided and the formulation prepared in Example 1 were used to prepare a laminated tooth whitening system, as follows. Initially, the formulation prepared in Example 1 was pressed between two polyethylene terephthalate (PET) release liners using shim plates (10 mil) and 40,000 lb force, to provide the inner tooth whitening layer of the laminated system (i.e., the tooth-contacting layer). One of the release liners was removed. The mixture prepared in this example was then pressed in the same manner to provide the outer layer of the tooth whitening system. The outer layer was then placed over the inner layer, and the release liner adjacent to the inner layer was removed. A layer of a nonwoven material (polyamide, obtained from Spunfab) was then placed against the inner layer, followed by a release liner. A release liner was then placed over the outer layer as well. The laminate so provided was pressed again at 40,000 lb, and the release liner adjacent to the outer layer was removed.

Individual strips were die cut using a Champion Die SR-1700-007.

### Example 3

The in vitro release of hydrogen peroxide from a tooth whitening system of the invention was compared with the hydrogen peroxide release profile exhibited by a commercial product, Crest Whitestrips™ (a product of the Procter & Gamble Co., Cincinnati, Ohio and referred to as the “Crest product”). The Crest product contains 5.3% hydrogen peroxide in a Carbopol 956 gel on a thin polyethylene film. The tooth whitening system of the invention was prepared by extruding the formulation of Example 1 onto a polyethylene backing to provide a tooth whitening layer approximately 0.35″ thick. The formulations were allowed to release peroxide in water, and the amount of peroxide released was measured at ten-minute intervals using standard analytical techniques.

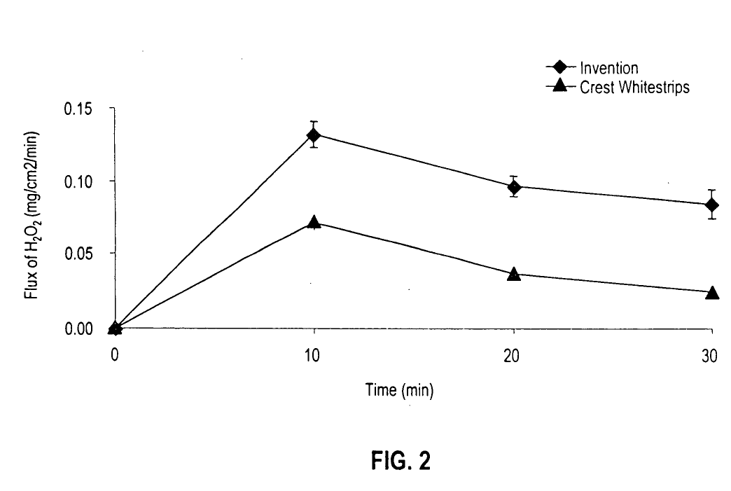

The flux of hydrogen peroxide released (in mg/cm²/min) was plotted for each system evaluated. As may be seen in the graph of FIG. 2, each of the systems exhibited maximum flux after ten minutes. At each measurement point, however, the flux of hydrogen peroxide released from the system of the invention was 2 to 3 times greater than the flux of hydrogen peroxide released from the Crest product.

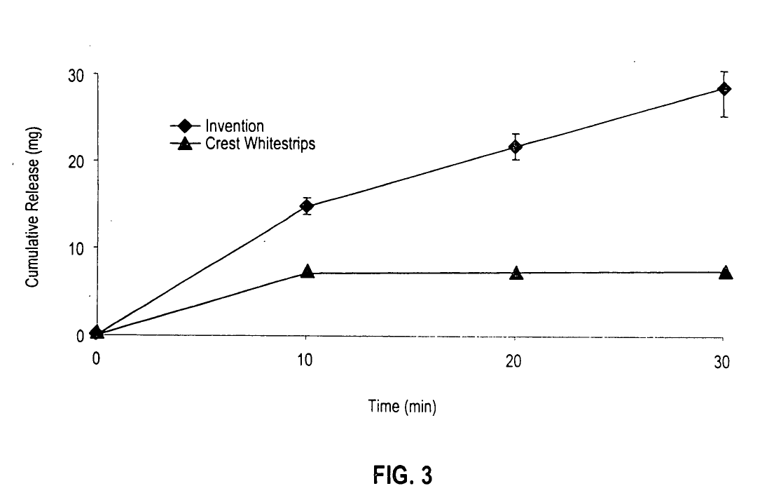

In FIG. 3, the cumulative amount of hydrogen peroxide released (in mg) at each measurement point was plotted for each system evaluated. The cumulative amount of hydrogen peroxide released from the Crest product remained constant, at approximately 7 mg, while the cumulative amount released from the system of the invention increased from approximately 15 mg at 10 minutes to approximately 22 mg at 20 minutes, to approximately 28 mg at 30 minutes.

### Example 4

The in vitro release of hydrogen peroxide from a second tooth whitening system of the invention was compared with the hydrogen peroxide release profile exhibited by the Crest product evaluated in Example 3. In this example, the tooth whitening system of the invention was that prepared in Example 2, having a Eudragit RS PO backing layer instead of a polyethylene backing. The evaluation method was the same as that of Example 3.

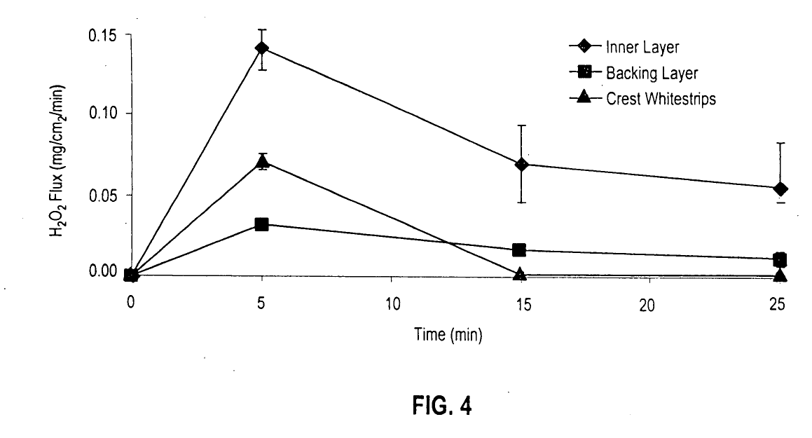

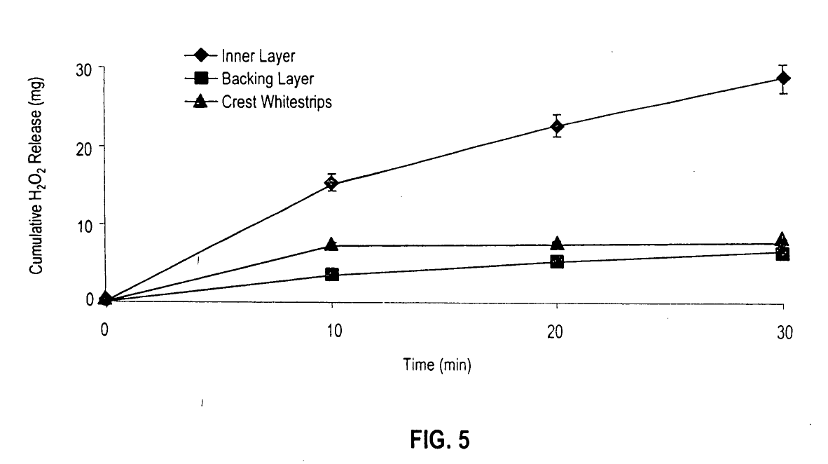

The graphs of FIG. 4 and FIG. 5 respectively illustrate the flux and cumulative amounts of hydrogen peroxide released from the inner, tooth-contacting layer of the inventive system, the outer backing layer of the inventive system, and the Crest product. The flux and release profiles are similar to the profiles obtained in Example 3.

### Example 5

In vivo evaluation: Four individuals participated in this study, applying the system of Example 2 to the lower teeth for 30 minutes. All four individuals reported that the initial adhesion was good, that adhesion during wear was good, and that the product was easily removed.

### Example 6

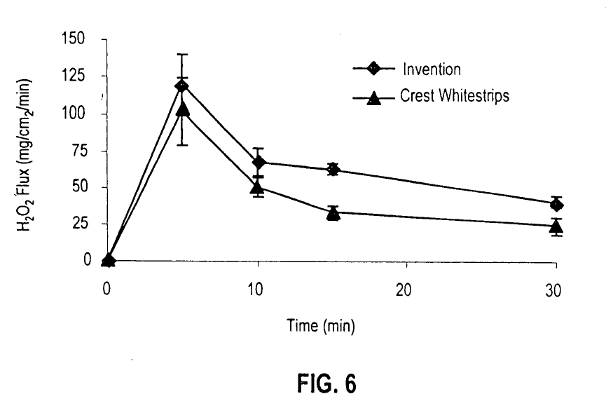

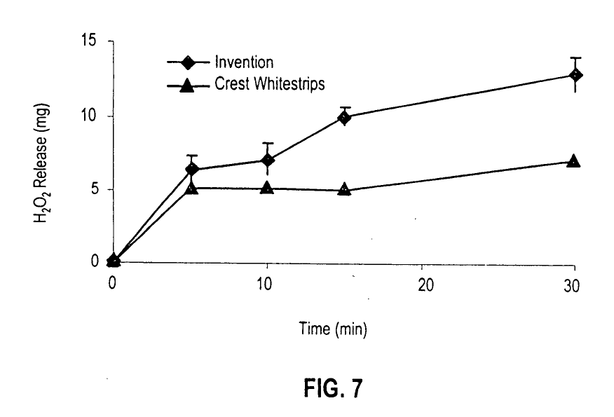

In vivo release: The system prepared in Example 2 was applied as a maxillary tooth whitening strip to each of four volunteers and removed at 5, 10, 15, and 30 minutes. The residual amount of hydrogen peroxide was evaluated using potassium permanganate titration. The hydrogen peroxide flux and cumulative hydrogen peroxide released were evaluated and plotted in FIGS. 6 and 7.

We claim:

1. In a tooth whitening composition comprising at least one tooth whitening agent, the improvement comprising incorporating a mixture of tooth whitening agents into the composition wherein a first whitening agent releases peroxide anion gradually upon contact with moisture and produces an alkaline pH, and a second whitening agent releases peroxide rapidly upon contact with moisture.

2. A tooth whitening composition comprising an admixture of:
a first whitening agent that is inert in a dry environment but activated upon contact with moisture to release hydrogen peroxide and produce an alkaline pH;
a second whitening agent that is inert in a dry environment but activated upon contact with aqueous base; and
a water-swellable, water-insoluble polymer.

3. The tooth whitening composition of claim 2, wherein the first tooth whitening agent is an addition compound of (a) a salt of an oxyanion, and (b) hydrogen peroxide.

4. The tooth whitening composition of claim 3, wherein the salt of the oxyanion is a carbonate salt.

5. The tooth whitening composition of claim 4, wherein the first tooth whitening agent is carbamide peroxide.

6. The tooth whitening composition of claim 5, wherein the second tooth whitening agent is sodium percarbonate.

7. The tooth whitening composition of claim 2, wherein the water-swellable, water-insoluble polymer is a cellulose ester.

8. The tooth whitening composition of claim 2, wherein the water-swellable, water-insoluble polymer is an acrylate-based polymer.

9. The tooth whitening composition of claim 8, wherein the acrylate-based polymer is a polymer or copolymer of acrylic acid, methacrylic acid, acrylate, methyl acrylate, ethyl acrylate, methyl methacrylate, ethyl methacrylate, a dialkylaminoalkyl acrylate, a dialkylaminoalkyl methacrylate, a trialkylammonioalkyl acrylate, and/or a trialkylammonioalkyl methacrylate.

10. The tooth whitening composition of claim 9, wherein the acrylate-based polymer is a polymer or copolymer of acrylic acid, methacrylic acid, methyl methacrylate, ethyl methacrylate, 2-dimethylaminoethyl methacrylate, and/or trimethylammonioethyl methacrylate chloride.

11. The tooth whitening composition of claim 2, comprising an admixture of at least two water-swellable, water-insoluble polymers.

12. The tooth whitening composition of claim 11, wherein a first water-swellable, water-insoluble polymer is cationic, a second water-swellable, water-insoluble polymer is anionic, and the polymers are ionically associated with each other to form a polymer matrix.

13. The tooth whitening composition of claim 12, wherein the cationic polymer is an acrylate-based polymer with pendant quaternary ammonium groups, and the anionic polymer is an ionized acrylic acid or methacrylic acid polymer.

14. The tooth whitening composition of claim 2, wherein the cationic polymer is a copolymer of trimethylammonioethyl methacrylate chloride and methyl methacrylate.

15. The tooth whitening composition of claim 2, further including a crosslinked hydrophilic polymer.

16. The tooth whitening composition of claim 15, wherein the hydrophilic polymer is covalently crosslinked.

17. The tooth whitening composition of claim 15, wherein the hydrophilic polymer is crosslinked via hydrogen bonding by a complementary oligomer.

18. The tooth whitening composition of claim 15, wherein the hydrophilic polymer is selected from the group consisting of poly(N-vinyl lactams), poly(N-vinyl amides), poly(N-alkylacrylamides), polyvinyl alcohol, polyvinylamine, and copolymers and blends thereof.

19. The tooth whitening composition of claim 18, wherein the hydrophilic polymer is selected from the group consisting of poly(N-vinyl lactams), poly(N-vinyl amides), poly(N-alkylacrylamides), and copolymers and blends thereof.

20. The tooth whitening composition of claim 19, wherein the hydrophilic polymer is a poly(N-vinyl lactam).

21. The tooth whitening composition of claim 20, wherein the hydrophilic polymer is a poly(N-vinyl lactam) homopolymer.

22. The tooth whitening composition of claim 21, wherein the poly(N-vinyl lactam) is selected from the group consisting of polyvinyl pyrrolidone, polyvinyl caprolactam, and blends thereof.

23. The tooth whitening composition of claim 22, wherein the poly(N-vinyl lactam) is polyvinyl pyrrolidone.

24. The tooth whitening composition of claim 15, wherein the hydrophilic polymer has a number average molecular weight in the range of approximately 100,000 to 2,000,000.

25. The tooth whitening composition of claim 24, wherein the hydrophilic polymer has a number average molecular weight in the range of approximately 500,000 to 1,500,000.

26. The tooth whitening composition of claim 17, wherein the complementary oligomer has a molecular weight in the range of about 45 to 800.

27. The tooth whitening composition of claim 26, wherein the complementary oligomer has a molecular weight in the range of about 45 to 600.

28. The tooth whitening composition of claim 27, wherein the complementary oligomer has a molecular weight in the range of about 300 to 600.

29. The tooth whitening composition of claim 17, wherein the complementary oligomer is selected from the group consisting of polyalcohols, monomeric and oligomeric alkylene glycols, polyalkylene glycols, carboxyl-teminated polyalkylene glycols, amino-terminated polyalkylene glycols, ether alcohols, alkane diols and carbonic diacids.

30. The tooth whitening composition of claim 29, wherein the complementary oligomer is selected from the group consisting of polyalkylene glycols and carboxyl-terminated polyalkylene glycols.

31. The tooth whitening composition of claim 29, wherein the complementary oligomer is polyethylene glycol.

32. The tooth whitening composition of claim 31, wherein the complementary oligomer is polyethylene glycol 400.

33. The tooth whitening composition of claim 15, further including a low molecular weight plasticizer.

34. The tooth whitening composition of claim 33, wherein the low molecular weight plasticizer is selected from the group consisting of dialkyl phthalates, dicycloalkyl phthalates, diaryl phthalates, mixed alkyl-aryl phthalates, alkyl phosphates, aryl phosphates, alkyl citrates, citrate esters, alkyl adipates, dialkyl tartrates, dialkyl sebacates, dialkyl succinates, alkyl glycolates, alkyl glycerolates, glycol esters, glycerol esters, and mixtures thereof.

35. The tooth whitening composition of claim 34, wherein the low molecular weight plasticizer is selected from the group consisting of dimethyl phthalate, diethyl phthalate, dipropyl phthalate, di(2-ethylhexyl)phthalate, di-isopropyl phthalate, diamyl phthalate, dicapryl phthalate, tributyl phosphate, trioctyl phosphate, tricresyl phosphate, triphenyl phosphate, trimethyl citrate, triethyl citrate, tributyl citrate, acetyl triethyl citrate, trihexyl citrate, dioctyl adipate, diethyl adipate, di(2-methylethyl)adipate, dihexyl adipate, diethyl tartrate, dibutyl tartrate, diethyl sebacate, dipropyl sebacate, dinonyl sebacate, diethyl succinate, dibutyl succinate, glycerol diacetate, glycerol triacetate, glycerol monolactate diacetate, methyl phthalyl ethyl glycolate, butyl phthalyl butyl glycolate, ethylene glycol diacetate, ethylene glycol dibutyrate, triethylene glycol diacetate, triethylene glycol dibutyrate, triethylene glycol dipropionate, and mixtures thereof.

36. The tooth whitening composition of claim 15, wherein the hydrophilic polymer represents less than 10 wt. % of the composition.

37. A tooth whitening composition comprising an admixture of:
a tooth whitening agent that is inert in a dry environment but activated in the presence of moisture; and
at least two water-swellable, water-insoluble polymers, wherein a first water-swellable, water-insoluble polymer is cationic, a second water-swellable, water-insoluble polymer is anionic, and the polymers are ionically associated with each other to form a polymer matrix.

38. The tooth whitening composition of claim 37, wherein the cationic polymer is an acrylate-based polymer with pendant quaternary ammonium groups, and the anionic polymer is an ionized acrylic acid or methacrylic acid polymer.

39. A tooth whitening composition comprising:
1.5 wt. % to 30 wt. % of a hydrophilic polymer composition composed of (a) a covalently crosslinked hydrophilic polymer, and/or (b) a blend of a hydrophilic polymer and a complementary oligomer capable of hydrogen bonding thereto;
40. wt. % to 90 wt. % of at least one water-swellable, water-insoluble polymer; and
at least one tooth whitening agent.

40. The tooth whitening composition of claim 39, wherein:
the hydrophilic polymer is selected from poly(N-vinyl lactams), poly(N-vinyl amides), poly(N-alkylacrylamides), polyvinyl alcohol, polyvinylamine, and copolymers thereof;
the complementary oligomer is selected from polyalcohols, monomeric and oligomeric alkylene glycols, polyalkylene glycols, carboxyl-teminated polyalkylene glycols, amino-terminated polyalkylene glycols, ether alcohols, alkane diols and carbonic diacids; and
the at least one water-swellable, water-insoluble polymer is an acrylate-based polymer.

41. The tooth whitening composition of claim 40, wherein:
the hydrophilic polymer is a poly(N-vinyl lactam);
the complementary oligomer is selected from the group consisting of polyethylene glycol and carboxyl-terminated polyethylene glycol; and
the acrylate-based polymer is a polymer or copolymer of acrylic acid, methacrylic acid, methyl methacrylate, ethyl methacrylate, 2-dimethylaminoethyl methacrylate, and/or trimethylammonioethyl methacrylate chloride.

42. The tooth whitening composition of claim 39, comprising an admixture of at least two water-swellable, water-insoluble polymers.

43. The tooth whitening composition of claim 42, wherein a first water-swellable, water-insoluble polymer is cationic, a second water-swellable, water-insoluble polymer is anionic, and the polymers are ionically associated with each other to form a polymer matrix.

44. The tooth whitening composition of claim 43, wherein the cationic polymer is an acrylate-based polymer with pendant quaternary ammonium groups, and the anionic polymer is an ionized acrylic acid or methacrylic acid polymer.

45. The tooth whitening composition of claim 39, comprising:
up to 10 wt. % of a blend of a hydrophilic polymer and a complementary oligomer capable of hydrogen bonding thereto;
at least 60 wt. % of at least one water-swellable, water-insoluble polymer; and
at least one tooth whitening agent.

46. A tooth whitening strip comprising a flexible strip of material in contact with the tooth whitening composition of any one of claims 1, 2, 37, or 39.

47. The tooth whitening strip of claim 46, further including a removable release liner covering the tooth whitening composition and preventing exposure of the composition to air.

48. A flexible, laminated tooth whitening strip, comprising:
a permeable outer layer that provides the outer surface of the strip following application to the teeth, the outer layer comprised of a relatively hydrophobic polymer and containing 1.0 to 30.0 wt. % of at least one tooth whitening agent; and
an interior whitening agent layer composed of a polymeric matrix containing 1.0 to 50.0 wt. % of at least one tooth whitening agent, the interior layer capable of adhering to the teeth in the presence of moisture.

49. The tooth whitening strip of claim 48, wherein the interior whitening agent layer comprises a mixture of a first tooth whitening agent that releases peroxide anion gradually upon contact with moisture and produces an alkaline pH, and a second tooth whitening agent that releases peroxide rapidly upon contact with moisture in the presence of base.

50. The tooth whitening strip of claim 48, wherein the interior whitening agent layer comprises an admixture of:
a first tooth whitening agent that is inert in a dry environment but activated upon contact with moisture to release hydrogen peroxide and produce an alkaline pH;
a second tooth whitening agent that is inert in a dry environment but activated upon contact with aqueous base; and
a water-swellable, water-insoluble polymer.

51. The tooth whitening strip of claim 48, wherein the interior whitening agent layer comprises at least two water-swellable, water-insoluble polymers, wherein a first water-swellable, water-insoluble polymer is cationic, a second water-swellable, water-insoluble polymer is anionic, and the polymers are ionically associated with each other to form a polymer matrix.

52. The tooth whitening strip of claim 48, wherein the interior whitening agent layer comprises an admixture of:
1.5 wt. % to 25 wt. % of a hydrophilic polymer composition composed of (a) a covalently crosslinked hydrophilic polymer, and/or (b) a blend of a hydrophilic polymer and a complementary oligomer capable of hydrogen bonding thereto;
45. wt. % to 90 wt. % of at least one water-swellable, water-insoluble polymer; and
the at least one tooth whitening agent.

53. The tooth whitening strip of claim 48, wherein the interior whitening agent layer is bisected into two separate layers by a nonwoven layer.

54. The tooth whitening strip of claim 48, further including a removable release liner covering the interior whitening agent layer and preventing exposure of the layer to air.

55. A packaged, anhydrous tooth whitening system comprising the tooth whitening strip of claim 48 in a moisture-free sealed pouch.
